(19) United States
(12) Patent Application Publication
(10) Pub. No.: US 2018/0177899 A1
(43) Pub. Date: Jun. 28, 2018

(54) HYDROPHILIC PARTICLES, METHOD FOR PRODUCING THE SAME, AND CONTRAST AGENT UTILIZING SAME

(71) Applicants: Korea Basic Science Institute, Cheongju-si, Chungcheongbuk-do (KR); Korea Research Institute Of Chemical Technology, Daejeon (KR)

(72) Inventors: Kwan Soo HONG, Cheongju-si (KR); Hye Sun PARK, Sejong (KR); Sang Hwan NAM, Sejong (KR); Yung Doug SUH, Seoul (KR)

(73) Assignees: Korea Basic Science Institute, Cheongju-si, Chungcheongbuk-do (KR); Korea Research Institute Of Chemical Technology, Daejeon (KR)

(21) Appl. No.: 15/580,977

(22) PCT Filed: May 19, 2016
(86) PCT No.: PCT/KR2016/005323

§ 371 Date: Dec. 8, 2017

(30) Foreign Application Priority Data
Jun. 10, 2015 (KR) 10-2015-0081804

(51) Int. Cl. A61K 49/18

(52) U.S. Cl. CPC A61K 49/1821

(57) ABSTRACT

Provided are a hydrophilic particle, a method for manufacturing the same, and a contrasting agent using the same. More specifically, the hydrophilic particle according to the inventive concept may include a hydrophobic particle, and an amphiphilic organic dye directly absorbed on a surface of the hydrophobic particle. In this case, the hydrophobic particle includes a center particle, and a hydrophobic ligand covering a surface of the center particle, and the amphiphilic organic dye may be combined to the hydrophobic ligand by a hydrophobic interaction. The hydrophilic particle may have a surface zeta potential lower than a surface zeta potential of the amphiphilic organic dye.

### CROSS-REFERENCE TO RELATED APPLICATIONS

This application is a national phase under 35 U.S.C. § 371 of PCT International Application No. PCT/KR2016/005323 which has an International filing date of May 19, 2016, which claims priority to Korean Application No. 10-2015-0081804, filed Jun. 10, 2015, the entire contents of each of which are hereby incorporated by reference.

### TECHNICAL FIELD

The present invention disclosed herein relates to a hydrophilic particle, the phase of which is converted by using an amphiphilic organic dye, a method for manufacturing the same, and a contrasting agent using the same.

### BACKGROUND ART

Nanoparticles have been extensively studied for scientific interest and potential applications due to the unique electrical, magnetic, and optical properties, and various functionalities thereof. The application of nanoparticles to the biomedical field have been attracting considerable attention thereto since nanoparticles are expected to improve medical diagnosis and treatment.

For the practical application of nanoparticles to the biomedical field, nanoparticles having both magnetic and fluorescence properties are needed in vivo in vitro applications. From this point of view, studies on multilayer nanoparticles combining magnetic nanoparticles and organic/inorganic phosphors are actively conducted. As a magnetic nanoparticle, a gadolinium nanoparticle which is a paramagnetic material is currently widely used clinically, and an iron oxide-based nanoparticle which is a superparamagnetic material is known to be able to be used as a contrasting agent using MRI.

However, materials constituting the core of such multilayer nanoparticles are mostly heavy metals, and thus, for biomedical applications, a processing for modifying the surface of a nanoparticle is needed. For example, a method of increasing the biocompatibility by introducing a silica layer on the surface of the nanoparticle is representative.

### DISCLOSURE OF THE INVENTION

### Technical Problem

The present invention provides a hydrophilic particle using an amphiphilic organic dye without surface modification.

The present invention also provides a method for manufacturing a hydrophilic particle, the method including a phase conversion method using an amphiphilic organic dye as an interface material.

The present invention also provides a contrasting agent including the hydrophilic particle.

### Technical Solution

A hydrophilic particle according to the inventive concept may include a hydrophobic particle, and an amphiphilic organic dye directly absorbed on a surface of the hydrophobic particle. In this case, the hydrophobic particle includes a center particle, and a hydrophobic ligand covering a surface of the center particle, and the amphiphilic organic dye may be combined with the hydrophobic ligand by a hydrophobic interaction. The hydrophilic particle may have a surface zeta potential lower than a surface zeta potential of the amphiphilic organic dye.

In an embodiment, the center particle includes a transition metal oxide, and the hydrophobic ligand may include a fatty acid.

In an embodiment, the transition metal oxide may be selected from the group consisting of iron oxide, manganese oxide, titanium oxide, nickel oxide, cobalt oxide, zinc oxide, ceria, and gadolinium oxide.

In an embodiment, the fatty acid may be selected from the group consisting of oleic acid, laurate acid, palmitic acid, linoleic acid, and stearic acid.

In an embodiment, the center particle is an up-conversion particle, and the hydrophobic ligand may include a fatty acid

In an embodiment, the up-conversion particle may be selected from the group consisting of NaYF₄:Yb³⁺,Er³⁺, NaYF₄:Yb³⁺,Tm³⁺, NaGdF₄:Yb³⁺,Er³⁺, NaGdF₄:Yb³⁺,Tm³⁺, NaYF₄:Yb³⁺,Er³⁺/NaGdF₄, NaYF₄:Yb³⁺,Tm³⁺/NaGdF₄, NaGdF₄:Yb³⁺,Tm³⁺/NaGdF₄, and NaGdF₄:Yb³⁺,Er³⁺/NaGdF₄.

In an embodiment, the fatty acid may be selected from the group consisting of oleic acid, laurate acid, palmitic acid, linoleic acid, and stearic acid.

In an embodiment, the amphiphilic organic dye may be selected from the group consisting of rhodamine, BODIPY, Alexa Fluor, fluorescein, cyanine, phtahlocyanine, an azo-group dye, a ruthenium-based dye, and derivatives thereof.

In an embodiment, the amphiphilic organic dye may include, in the molecule thereof, a hydrophilic group selected from the group consisting of a carboxyl group, a sulfonic acid group, a phosphonic acid group, an amine group, and an alcohol group, and a hydrophobic group selected from the group consisting of an aromatic hydrocarbon and an aliphatic hydrocarbon.

In an embodiment, the surface zeta potential of the amphiphilic organic dye may be a value measured when the amphiphilic organic dye is present alone.

In an embodiment, the surface zeta potential of the hydrophilic particle may be a negative charge.

In an embodiment, an average diameter of the hydrophilic particle may be greater than an average diameter of the hydrophobic particle.

A method for manufacturing a hydrophilic particle according to another inventive concept may include preparing a hydrophobic particle dispersed in an organic phase, and mixing the hydrophobic particle in the organic phase with an amphiphilic organic dye in an aqueous phase to form a hydrophilic particle. In this case, the amphiphilic organic dye may be directly absorbed on a surface of the hydrophobic particle to phase-convert the hydrophobic particle to the hydrophilic particle dispersed in the aqueous phase

In an embodiment, the mixing of the hydrophobic particle and the amphiphilic organic dye may include adding the hydrophobic particle in the organic phase to the amphiphilic organic dye in the aqueous phase, and ultrasonicating the mixture of the hydrophobic particle and the amphiphilic organic dye to form a water-in-oil (O/W) emulsion.

In an embodiment, the organic phase may include an organic solvent selected from the group consisting of chloroform, cyclohexane, hexane, heptane, octane, isooctane, nonane, decane, and toluene.

In an embodiment, the hydrophobic particle includes a hydrophobic ligand on a surface thereof, and the amphiphilic organic dye may be combined with the hydrophobic ligand by a hydrophobic interaction

In an embodiment, the method may further include, after forming the hydrophilic particle, evaporating the organic solvent constituting the organic phase.

A contrasting agent according to another inventive concept may include a hydrophilic particle. In this case, the hydrophilic particle may include a hydrophobic particle, and an amphiphilic organic dye directly adsorbed on a surface of the hydrophobic particle. A surface zeta potential of the hydrophilic particle may be lower than a surface zeta potential of the amphiphilic organic dye.

In an embodiment, the contrasting agent may be used for magnetic resonance imaging, optical imaging, or magnetic resonance imaging and optical imaging.

### Advantageous Effects

A hydrophilic particle according to the inventive concept may have two contrasting functions through the combination of an amphiphilic organic dye positioned on the surface thereof, and a center particle. In addition, a hydrophilic particle according to the inventive concept has high biocompatibility and may increase the stability of an organic dye combined to the surface thereof. Furthermore, a method for manufacturing a hydrophilic particle according to the inventive concept may be performed simply and quickly through a phase conversion method without surface modification of a particle and a surfactant.

### BRIEF DESCRIPTION OF THE DRAWINGS

FIG. 1A is a cross-sectional view schematically showing a hydrophilic particle according to embodiments of the inventive concept. FIG. 1B is an enlarged cross-sectional view of the region M of FIG. 1A;

FIG. 2 is a cross-sectional view schematically showing a method for manufacturing a hydrophilic particle according to embodiments of the inventive concept;

FIGS. 3A and 3B show a TEM image and a size analysis result of iron oxide nanoparticles (IONP) dispersed in an organic solvent;

FIG. 4 shows the process of purification using the magnetic force of an iron oxide nanoparticle dispersed in an aqueous phase;

FIGS. 5A and 5B show a TEM image and a size analysis result of indocyanine green coated iron oxide nanoparticles (ICG coated IONP) dispersed in an aqueous phase;

FIG. 6 is a comparative analysis result of the surface charges of an iron oxide nanoparticle coated with indocyanine green (IONP-ICG) and dispersed in an aqueous phase, and a pure indocyanine green (ICG) solution;

FIG. 7 is a FT-IR analysis result of indocyanine green (ICG), an iron oxide nanoparticle (IONP), and an iron oxide nanoparticle coated with indocyanine green (IONP-ICG);

FIGS. 8 and 9 respectively show a comparison analysis result of the absorbance spectrum and fluorescence spectrum of an iron oxide nanoparticle coated with indocyanine green (IONP-ICG) and dispersed in an aqueous phase, and a pure indocyanine green (ICG) solution;

FIG. 10 shows T2-weighted MR phantom images and r2 values in accordance with various concentrations of an iron oxide nanoparticle coated with indocyanine green dispersed in an aqueous phase;

FIG. 11 are an image of fluorescence signal and an MR image thereof, the signal appearing at the lymph node after the injection of iron oxide nanoparticles coated with indocyanine green into a sole a mouse;

FIGS. 12A and 12B show a TEM image and a size analysis result of up-conversion nanoparticles dispersed in an organic solvent;

FIGS. 13A and 13C are photoluminescence (PL) images (UCNP) of an up-conversion nanoparticle coated with indocyanine green.

FIGS. 13B and 13D are fluorescence images (ICG) of an up-conversion nanoparticle coated with indocyanine green;

FIGS. 14A to 14D are a single particle fluorescence images showing the optical stability of an up-conversion nanoparticle coated with indocyanine green.

### MODE FOR CARRYING OUT THE INVENTION

Objects, other objects, features, and advantages of the inventive concept described above may be understood easily by reference to the exemplary embodiments and the accompanying drawings. The inventive concept may, however, be embodied in different forms and should not be construed as limited to the embodiments set forth herein. Rather, these embodiments are provided so that this disclosure will be thorough and complete, and will fully convey the scope of the inventive concept to those skilled in the art.

In the accompanying drawings, the sizes, thicknesses, and the like of the structures are exaggerated for clarity of the inventive concept. Also, it will be understood that, although the terms first, second, etc. may be used herein to describe various elements, these elements should not be limited by these terms. These terms are only used to distinguish one element from another. Each embodiment described and exemplified herein also includes the complementary embodiment thereof. The term “and/or,” is used herein to include at least one of the elements listed before and after. Like reference numerals refer to like elements through the specification.

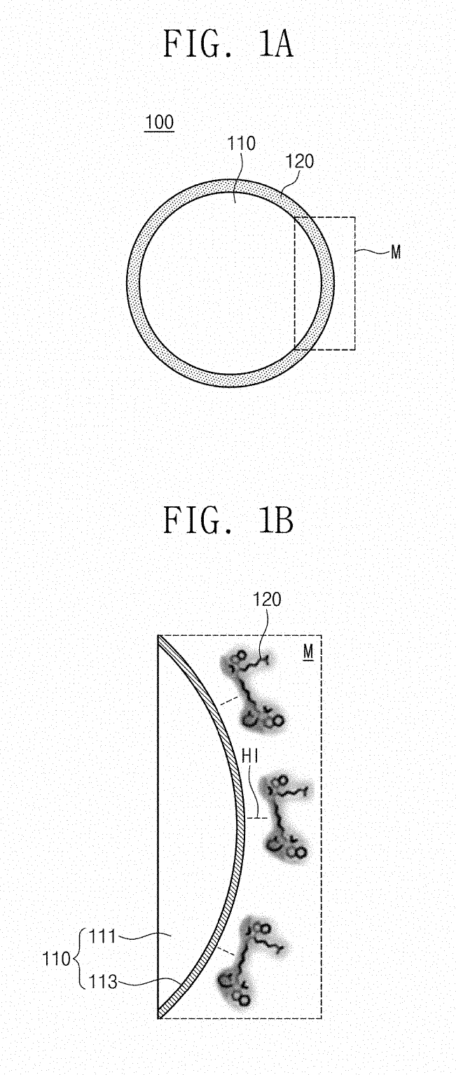

FIG. 1A is a cross-sectional view schematically showing a hydrophilic particle according to embodiments of the inventive concept. FIG. 1B is an enlarged cross-sectional view of the region M of FIG. 1A.

Referring to FIGS. 1A and 1B, a hydrophilic particle 100 including a hydrophobic particle 110 and an amphiphilic organic dye 120 may be provided. The amphiphilic organic dye 120 may be directly adsorbed on the surface of the hydrophobic particle 110. The adsorption may be caused by the physical interaction between the hydrophobic particle 110 and the amphiphilic organic dye 120.

The hydrophobic particle 110 may include a center particle 111, and a hydrophobic ligand 113 coated on the surface of the center particle 111. For example, the hydrophobic ligand 113 may form a single layer and cover the surface of the center particle 111. As another example, the hydrophobic ligand 113 may be uniformly or non-uniformly combined to the inside and to the surface of the center particle 111. The hydrophobic ligand 113 may impart hydrophobicity to the central particle 111. Thus, a plurality of hydrophobic particles 110 may be solely dispersed in an organic solvent without surfactant.

According to an embodiment of the inventive concept, the center particle 111 may be a transition metal oxide particle. That is, the particle center 111 may include one or more transition metal oxides selected from the group consisting of iron oxide, manganese oxide, titanium oxide, nickel oxide, cobalt oxide, zinc oxide, ceria and gadolinium oxide. The transition metal oxide, especially iron oxide, may have magnetic properties under an external magnetic field. When the external magnetic field is removed, the magnetism remaining in the transition metal oxide may disappear. Therefore, side effects due to the remaining magnetism may be reduced. Furthermore, the transition metal oxide may be biodegraded in the body, so that the biocompatibility thereof may be excellent. The transition metal oxide may be used as a cell marking material for magnetic resonance imaging (MRI) tracking of therapeutic cells.

In another embodiment, the center particle 111 may be an up-conversion particle. The up-conversion particle may be a particle capable of emitting a visible ray when a near infrared ray is irradiated thereon. The up-conversion particle may be a particle which is an inorganic host doped with a rare earth element. For example, the up-conversion particle may include one or more selected from the group consisting of NaYF₄:Yb³⁺,Er³⁺, NaYF₄:Yb³⁺,Tm³⁺, NaGdF₄:Yb³⁺,Er³⁺, NaGdF₄:Yb³⁺,Tm³⁺, NaYF₄:Yb³⁺,Er³⁺/NaGdF₄, NaYF₄:Yb³⁺,Tm³⁺/NaGdF₄, NaGdF₄:Yb³⁺,Tm³⁺/NaGdF₄, and NaGdF₄:Yb³⁺,Er³⁺/NaGdF₄.

Furthermore, since the surface thereof exhibits hydrophobicity, the hydrophobic particle 110 is not particularly limited as long as the particle may be dispersed in the organic solvent.

The hydrophobic ligand 113 may include a fatty acid. For example, the fatty acid may include at least one selected from the group consisting of oleic acid, lauric acid, palmitic acid, linoleic acid, and stearic acid.

The amphiphilic organic dye 120 may be an organic dye having both a hydrophilic group and a hydrophobic group in the molecule thereof. The hydrophilic group may be selected from the group consisting of a carboxyl group, a sulfonic acid group, phosphonic acid group, an amine group, and an alcohol group, and the hydrophobic group may be selected from the group consisting of an aromatic hydrocarbon and an aliphatic hydrocarbon. Specifically, the amphiphilic organic dye 120 may be a fluorescence organic dye, and may be selected from the group consisting of rhodamine, BODIPY, Alexa Fluor, fluorescein, cyanine, phtahlocyanine, an azo-group dye, a ruthenium-based dye, and derivatives thereof. For example, the amphiphilic organic dye 120 may include indocyanine green.

By a hydrophobic interaction (HI) between a hydrophobic group of the amphiphilic organic dye 120 and the hydrophobic ligand 113, the amphiphilic organic dye 120 may be combined with the hydrophobic ligand 113. Thus, as described above, the amphiphilic organic dye 120 may be directly adsorbed (or coated) on the surface of the hydrophobic particle 110.

The average diameter of the hydrophilic particle 100 may be 10 nm to 1000 nm. In this case, the average diameter of the hydrophilic particle 100 may be greater than the average diameter of the hydrophobic particle 110. In an embodiment of the inventive concept, the average diameter of the hydrophilic particle 100 may be greater than two times the average diameter of the hydrophobic particle 110.

The hydrophilic particle 100 may have a first surface zeta potential. When the amphiphilic organic dye 120 is present alone, the amphiphilic organic dye 120 may have a second surface zeta potential. In this case, the first surface zeta potential may be lower than the second surface zeta potential. That is, the hydrophilic particle 100 may have relatively negative charge properties compared to the amphiphilic organic dye 120 present alone. For example, the first surface zeta potential may be a negative charge, and may specifically be −100 mV to −10 mV.

As the amphiphilic organic dye 120 is adsorbed on the surface of the hydrophobic particle 110, the hydrophilic group of the amphiphilic organic dye 120 may be exposed to the outside more than the hydrophobic group. Accordingly, the hydrophilic group of the amphiphilic organic dye 120 may be relatively more distributed on the surface of the hydrophilic particle 100. Thus, the surface charge of the hydrophilic particle 100 may have relatively a negative value compared to the amphiphilic organic dye 120 present alone.

The hydrophilic particle 100 may be used as a contrasting agent through the amphiphilic organic dye 120 positioned on the surface thereof. In one embodiment, when the amphiphilic organic dye 120 is a fluorescence organic dye, fluorescence contrasting may be possible for the hydrophilic particle 100. Furthermore, the center particle 111 in the hydrophilic particle 100 may have a contrasting function. For example, when the center particle 111 includes a transition metal oxide, MR contrasting may be possible. In this case, the hydrophilic particle 100 may have two contrasting functions (MR contrasting and fluorescence contrasting), and therefore, may be utilized for in vivo and in vitro molecular imaging in the biomedical field. As another example, when the center particle 111 is an up-conversion particle, photoluminescence (PL) contrasting may be possible. In this case, the hydrophilic particle 100 may have two contrasting functions (PL contrasting and fluorescence contrasting).

In addition, since the hydrophilic particle 100 has hydrophilicity, the biocompatibility thereof may be excellent. Since the amphiphilic organic dye 120 is combined with the hydrophobic particle 110 by a hydrophobic interaction (HI), the fluorescence stability of the amphiphilic organic dye 120 may be increased.

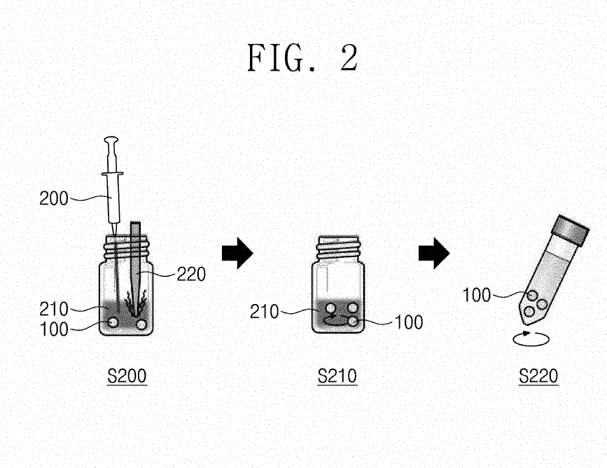

FIG. 2 is a cross-sectional view schematically showing a method for manufacturing a hydrophilic particle according to embodiments of the inventive concept.

Referring FIGS. 1A, 1B, and 2, by ultrasonicating a mixture of a first solution 200 and a second solution 210, an emulsion 220 may be formed S200. Specifically, first, the first solution 200 may be prepared. The first solution 200 may include the hydrophobic particles 110, and an organic solvent in which the hydrophobic particles 110 are dispersed. In other words, the hydrophobic particles 110 may be dispersed in an organic phase. Preparing the first solution 200 may include using various known methods such as a co-precipitation method, a thermal decomposition method, a hydrothermal synthesis method, or a microemulsion method. For example, the first solution 200 may be prepared by using the thermal decomposition method. When the thermal decomposition method is used, the fine size adjustment of the hydrophobic particles 110 is possible, and the size distribution of the hydrophobic particles 110 may be uniform, and the crystallinity of the hydrophobic particles 110 may be increased.

Each of the hydrophobic particles 110 may include a center particle 111, and a hydrophobic ligand 113 coated on the surface of the central particle 111. In one embodiment, the center particle 111 may be a transition metal oxide particle, and in another embodiment, the center particle 111 may be an up-conversion particle. However, the hydrophobic particle 110 is not particularly limited as long as the surface of the particle exhibits hydrophobicity, so that the particle may be dispersed in the organic solvent. The detailed description of the hydrophobic particle 110 may be the same as described above with reference to FIG. 1A and FIG. 1B. The organic solvent may be one or more selected from the group consisting of chloroform, cyclohexane, hexane, heptane, octane, isooctane, nonane, decane, and toluene, and is not particularly limited.

The second solution 210 may be prepared. The second solution 210 may be prepared by mixing the amphiphilic organic dye 120 and water. In other words, the amphiphilic organic dye 120 may be an aqueous phase. For example, the amphiphilic organic dye 120 may be a fluorescence organic dye having both a hydrophilic group and a hydrophobic group in the molecule thereof. The detailed description of the amphiphilic organic dye 120 may be the same as described above with reference to FIG. 1A and FIG. 1B.

The mixture may be prepared by adding the first solution 200 to the second solution 210. By using an ultrasonic tip apparatus, ultrasonicating of the mixture may be performed. The ultrasonicating may be performed for 10 seconds to 10 minutes. Thus, the first solution 200 and the second solution 210 is homogeneously mixed to form the oil-in-water (O/W) emulsion 220.

At the same time as the first solution 200 and the second solution 210 are mixed, the amphiphilic organic dye 120 may be directly adsorbed on the surface of the hydrophobic particle 110. Thus, the hydrophilic particle 100 which is the hydrophobic particle 110 coated with the amphiphilic organic dye 120 on the surface thereof may be formed. Specifically, by a hydrophobic interaction (HI) between a hydrophobic group of the amphiphilic organic dye 120 and the hydrophobic ligand 113, the amphiphilic organic dye 120 may be combined with the hydrophobic ligand 113. Without chemical reaction and only by ultrasonicating, the amphiphilic organic dye 120 may be directly adsorbed on the surface of the hydrophobic particle 110 by a physical interaction between the amphiphilic organic dye 120 and the hydrophobic particle 110.

By stirring the emulsion 220, the organic solvent may be evaporated S210. The stirring may be performed for 1 minute to 60 minutes. As a result, the hydrophilic particles 100 may be dispersed in an aqueous phase. Specifically, the hydrophilic particles 100 may be stably dispersed in an aqueous phase due to a hydrophilic group of the amphiphilic organic dye 120 present on the surface thereof. In other words, without surfactant, the phase of the hydrophobic particle 110 may be converted through the amphiphilic organic dye 120.

Next, the hydrophilic particles 100 may be purified S220. Performing the purification may include using centrifugation or using a magnetic force. For example, using centrifugation may include performing centrifugation on the emulsion 220, and removing supernatant. This process may be repeatedly performed until the amphiphilic organic dye 120 which is dispersed without being absorbed on the surface of the hydrophilic particle 100 is removed.

As another example, when the center particle 111 of the hydrophilic particle 100 is a transition metal oxide particle, the magnetic force may be used. Using the magnetic force may include adhering a powerful magnet to the emulsion 220, and then removing supernatant. This process may be repeatedly performed until the amphiphilic organic dye 120 which is dispersed without being absorbed on the surface of the hydrophilic particle 100 is removed.

A method for manufacturing the hydrophilic particle 100 according to the inventive concept may be performed simply and quickly by a phase conversion method using the amphiphilic organic dye 120 as an interface material, without surface modification of a particle, or surfactant.

Hereinafter, preferred experimental examples will be described in order to facilitate understanding of the inventive concept. It should be understood, however, that the following experimental examples are for illustrative purposes only and are not intended to limit the scope of the inventive concept.

### Experimental Example 1: Synthesis of an Iron Oxide Nanoparticle

36 g of iron-oleate (Fe-oleate, 40 mmol), 5.7 g of oleic acid (oleic acid, 20 mmol), and 200 g of octadecene (1-octadecene) were mixed. The mixture was stirred for 30 minutes under reduced pressure at room temperature to remove gas and water in the mixture. The mixture was heated to 320° C. at a heating rate of 3.3° C./min. At this time, the reduced pressure state was maintained up to 200° C., and at a temperature higher than 200° C., an inert atmosphere was maintained. After the mixture was stirred for 30 minutes at 320° C., a heater was removed, and the mixture was slowly cooled to room temperature. Thereafter, ethanol was added to the mixture in six times the volume of the mixture to precipitate the formed iron oxide nanoparticle. Then, supernatant was removed, and the iron oxide nanoparticle was separated to be redispersed in n-hexane. The concentration of the iron oxide nanoparticle in n-hexane was adjusted to be 10 mg/ml (Example 1).

The nanoparticle of Example 1 was dropped on a copper grid coated with carbon to prepare a sample, and a transmission electron microscope (TEM) image thereof was obtained with a high resolution electron microscope of 200 kV (Tecnai F20). Furthermore, the size of the nanoparticle of Example 1 was measured using a particle size surface charge analyzer (Zetasizer Nano-ZS, Otsuka).

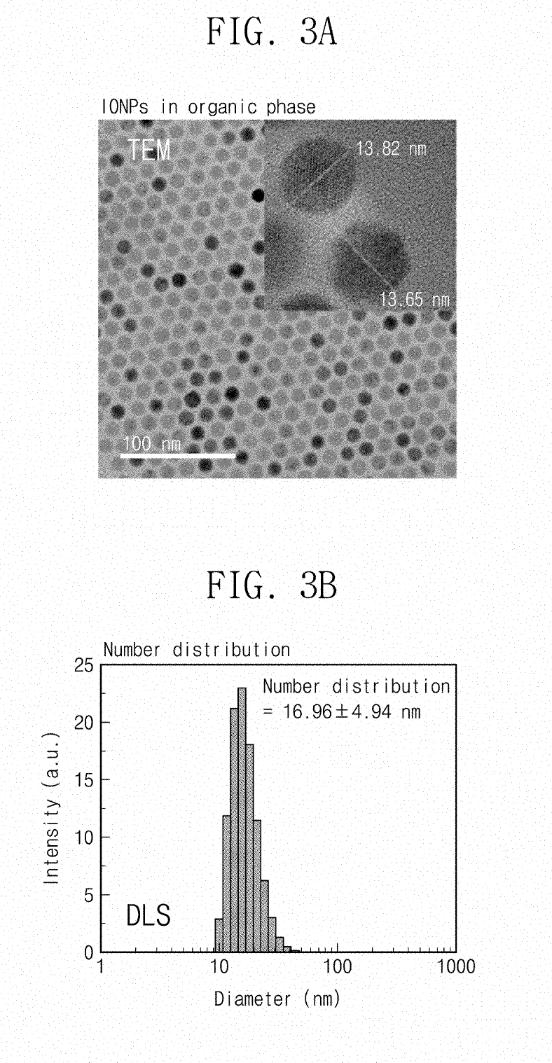

FIGS. 3A and 3B show a TEM image and a size analysis result of iron oxide nanoparticles (IONP) dispersed in an organic solvent.

Referring to FIG. 3A, a TEM image of the nanoparticle of Example 1 can be seen. It is confirmed that the nanoparticle of Example 1 has a very uniform shape and size with an average diameter of about 13 to 14 nm. Since the surface of the nanoparticle of Example 1 is coated with oleic acid, it is confirmed that the nanoparticle is very well dispersed in the organic solvent. However, the nanoparticle of Example 1 has the surface properties which prevent the nanoparticle from being dispersed at all in an aqueous phase.

Furthermore, referring to FIG. 3B, the size of the nanoparticle of Example 1 dispersed in n-hexane was measured to be about 17 nm by dynamic light scattering (DLS) analysis.

### Experimental Example 2: Preparation of an Iron Oxide Nanoparticle Coated with Indocyanine Green

1 ml of the iron oxide nanoparticle (Example 1) dispersed in n-hexane at a concentration of 10 mg/ml was taken, and then 9 ml of methanol was added thereto to precipitate the iron oxide nanoparticle. Then, supernatant was removed, and 1 ml of chloroform was added thereto to redisperse the iron oxide nanoparticle. After 2 mg of indocyanine green was dissolved in 4 ml of distilled water, 0.1 ml of the iron oxide nanoparticle dispersed in chloroform was added thereto, and tip ultrasonication was performed thereon for 1 minute to prepare a first emulsion solution. The emulsion solution was vigorously stirred for 5 minutes until all the chloroform therein was volatilized and removed

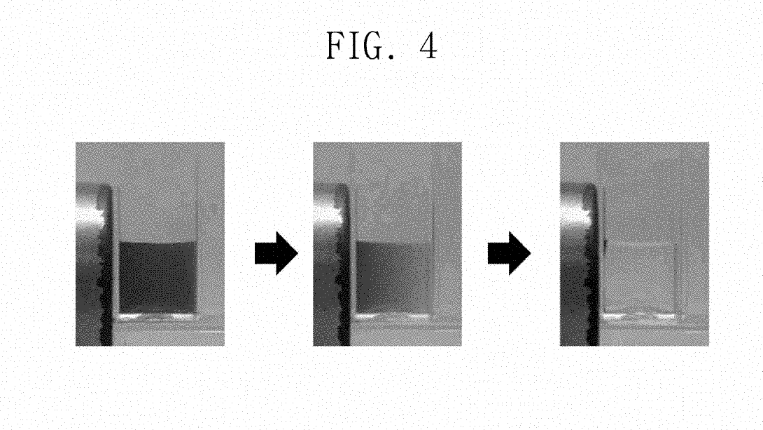

FIG. 4 shows the process of purification using the magnetic force of an iron oxide nanoparticle dispersed in an aqueous phase.

Referring to FIG. 4, the magnetic properties of the iron oxide nanoparticle was used to remove indocyanine green not adsorbed on the iron oxide nanoparticle. Specifically, the solution was held in close contact with a powerful magnet, and when the iron oxide nanoparticle was collected near the magnet, supernatant was removed, and 1 ml of distilled water was added thereto to redisperse the iron oxide nanoparticle. The process was repeated 4-5 times until the indocyanine green in the solution was removed so that the solution became transparent. The finally obtained iron oxide nanoparticle coated with indocyanine green was dispersed in 1 ml of distilled water (Example 2).

### Experimental Example 3: Analysis of the Characteristics of an Iron Oxide Nanoparticle Coated with Indocyanine Green

The nanoparticle of Example 2 was dropped on a copper grid coated with carbon to prepare a sample, and a transmission electron microscope (TEM) image thereof was obtained with a high resolution electron microscope of 200 kV (Tecnai F20). Furthermore, the size of the nanoparticle of Example 2 was measured using a particle size surface charge analyzer (Zetasizer Nano-ZS, Otsuka).

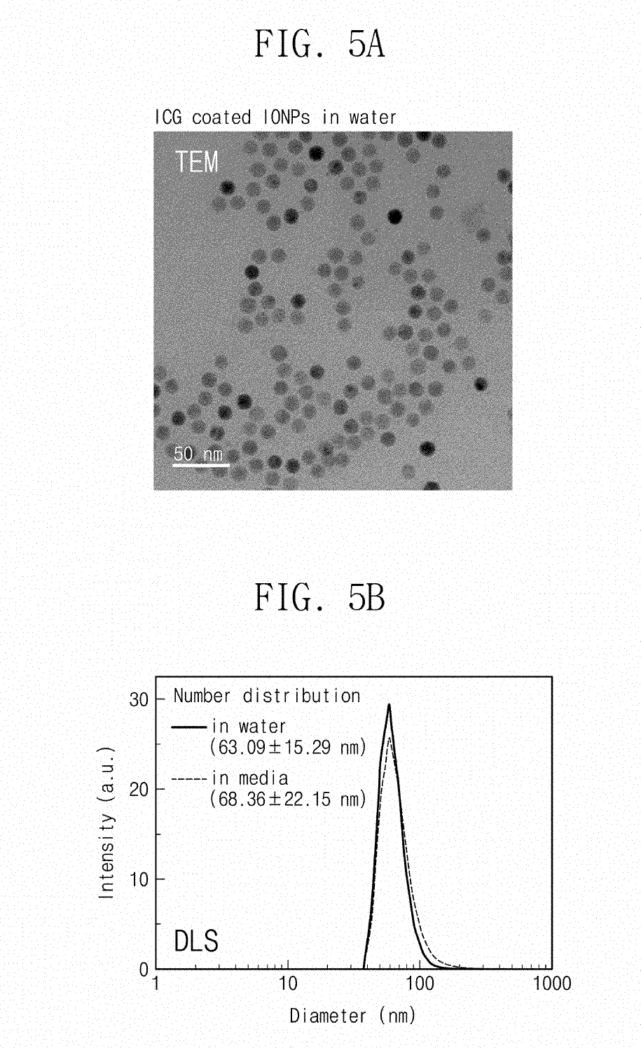

FIGS. 5A and 5B show a TEM image and a size analysis result of indocyanine green coated iron oxide nanoparticles (ICG coated IONP) dispersed in an aqueous phase.

Referring to FIG. 5A, it was confirmed that the nanoparticle of Example 2, unlike the iron oxide nanoparticle of Example 1, was well dispersed in the aqueous phase without an aggregation phenomenon. Due to the indocyanine green which is an amphiphilic organic dye, the surface of the iron oxide nanoparticle was converted to hydrophilic and therefore, the phase conversion thereof was achieved.

Referring to FIG. 5B, the size of the nanoparticle of Example 2 dispersed in the aqueous phase and cell media was measured to be about 63 nm and 69 nm, respectively.

The surface charge of the nanoparticle of Example 2 was measured using a particle size surface charge analyzer (Zetasizer Nano-ZS, Otsuka). In addition, the surface charge of a pure indocyanine green solution was measured using the particle size surface charge analyzer.

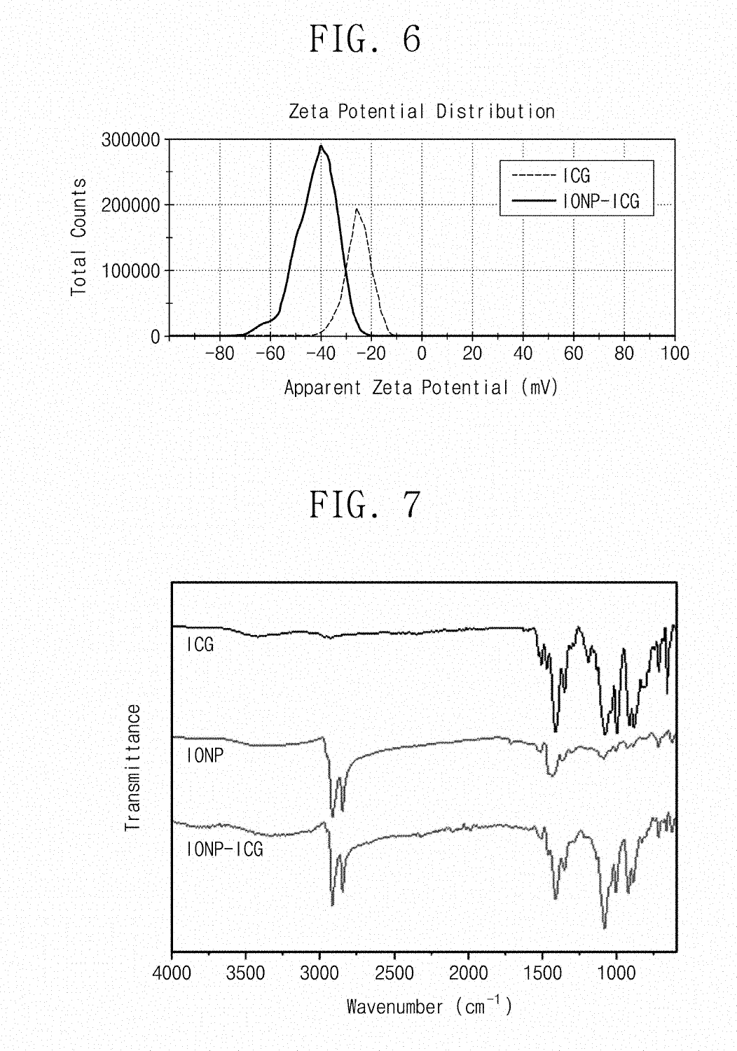

FIG. 6 is a comparative analysis result of the surface charges of an iron oxide nanoparticle coated with indocyanine green (IONP-ICG) and dispersed in an aqueous phase, and a pure indocyanine green (ICG) solution.

When indocyanine green is adsorbed on an iron oxide nanoparticle, a lipophilic group of the indocyanine green is absorbed on the surface of the nanoparticle, so that a hydrophilic group thereof is exposed on an aqueous phase, relatively. Therefore, a charge of the indocyanine green surrounding the nanoparticle may be a charge more negative than that in the pure indocyanine green solution. Referring to FIG. 6, when the surface charges of two solutions were measured and compared, the charge of the pure indocyanine green solution was measured to be −25.1 mV, and the charge of the nanoparticle of Example 2 was measured to be −41.6 mV. Therefore, it is confirmed that the surface charge of the nanoparticle of Example 2 is more negative than that of the pure indocyanine green.

Indocyanine green, the iron oxide nanoparticle of Example 1, and the nanoparticle coated with indocyanine green of Example 2 were prepared, each in a powder state. The FT-IR spectra thereof were measured using a surface reflection infrared spectrometer (ALPHA-P, Bruker). FIG. 7 is a FT-IR analysis result of indocyanine green (ICG), an iron oxide nanoparticle (IONP), and an iron oxide nanoparticle coated with indocyanine green (IONP-ICG).

The absorption and fluorescence spectra of the nanoparticle dispersed in the aqueous phase of Example 2, and the pure indocyanine green were measured using a UV-Vis spectrometer (UV-2600, shimadzu) and a fluorescence spectrometer (FS-2, Sinco). Furthermore, the nanoparticle dispersed in the aqueous phase of Example 2 was prepared in PCR tubes at various concentrations. The T2-weighted MR phantom images thereof were obtained, and each of the 1/T2 values thereof was obtained. Using the obtained 1/T2 values and the concentration ratio, the relaxivity value (r2) was calculated.

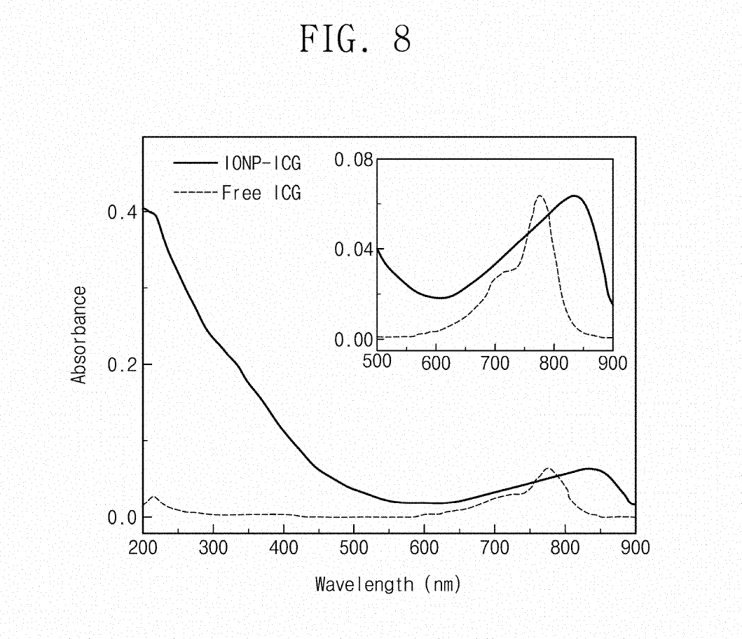

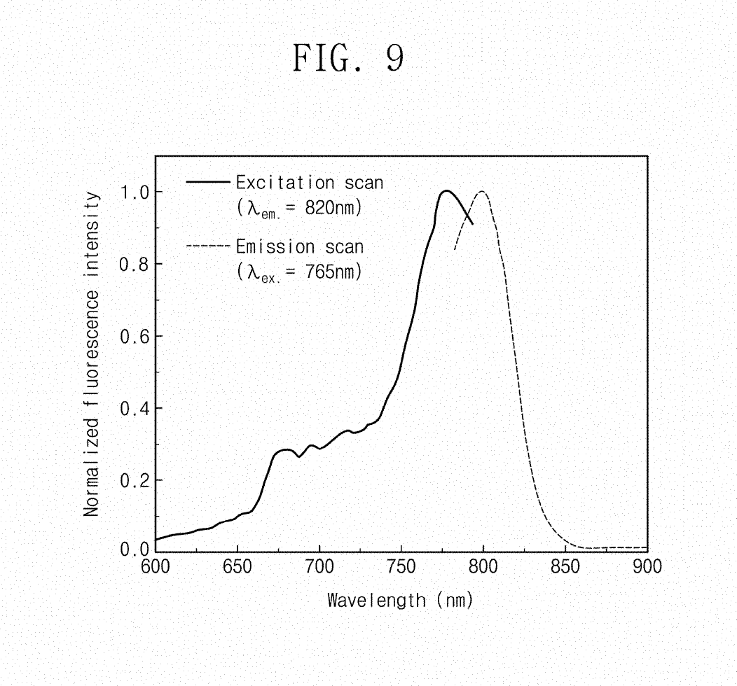

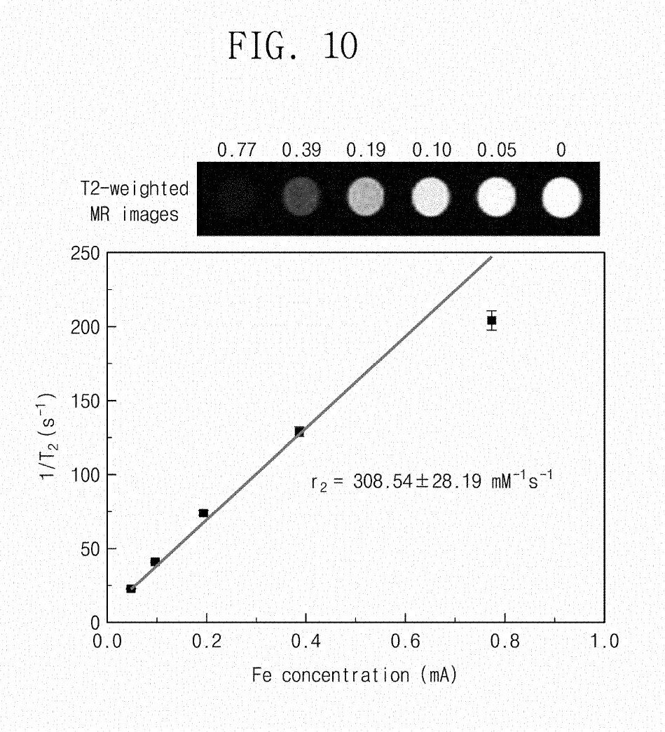

FIGS. 8 and 9 respectively show a comparison analysis result of the absorbance spectrum and fluorescence spectrum of an iron oxide nanoparticle coated with indocyanine green (IONP-ICG) and dispersed in an aqueous phase, and a pure indocyanine green (ICG) solution. FIG. 10 shows T2-weighted MR phantom images and r2 values in accordance with various concentrations of an iron oxide nanoparticle coated with indocyanine green dispersed in an aqueous phase;

Indocyanine green is an amphiphilic dye structurally having both a hydrophilic group and a hydrophobic group, so that, when injected into the blood, indocyanine green is capable of moving exhibiting very good adsorption properties on proteins present in the blood. That is, through a hydrophobic interaction of which a hydrophobic group of indocyanine green is absorbed and embedded on a hydrophobic portion of a protein, the indocyanine green may have good absorption properties. In this case, the absorbance spectrum of indocyanine green may be shifted in a long wavelength direction after being combined with a protein. Referring to FIG. 8, when the absorbance spectra of the nanoparticle of Example 2 and the pure indocyanine green solution were compared, it was confirmed that the absorption wavelength of the nanoparticle of the Example 2 was red-shifted to a long wavelength. That is, a hydrophobic portion of the indocyanine green is adsorbed and coated on the surface of the hydrophobic iron oxide nanoparticle coated with oleic acid.

Referring to FIG. 9, in the case of the fluorescence spectrum of the nanoparticle of Example 2, it is confirmed that, when excited to 765 nm, the near infrared fluorescence signal of 800 nm region is well displayed.

Referring to FIG. 10, it was confirmed the measured relaxivity r2 representing the MR contrasting capability of the nanoparticle of Example 2 was about 308 mM 1 s-1. From the result, it is confirmed that the nanoparticle of Example 2 has both characteristics of MR contrasting and near infrared fluorescence contrasting.

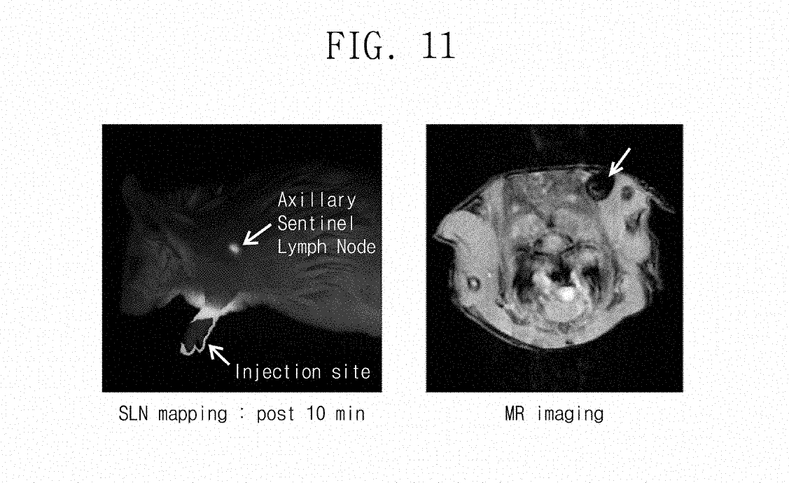

30 μl of the nanoparticle solution of Example 2 was injected into the front sole of a BALB/c mouse. A fluorescence signal obtained by irradiating 808 nm laser to the mouse was captured using an 830 nm long pass filter and an EM-CCD camera. In the same manner, a T2-weighted MR phantom image was obtained using a 4.7 T MRI (Bruker), and an MR image was obtained by cutting a lymph node. The results are shown in FIG. 11. FIG. 11 are an image of fluorescence signal and an MR image thereof, the signal appearing at the lymph node after the injection of iron oxide nanoparticles coated with indocyanine green into a sole a mouse.

### Experimental Example 4: Synthesis of an Up-Conversion Nanoparticle

779.4 mg of yttrium-oleate (Y-oleate, 0.78 mmol), 216.7 mg of ytterbium-oleate (Yb-oleate, 0.20 mmol), 21.6 mg of erbium-oleate (Er-oleate, 0.02 mmol), 8 ml of oleic acid, and 200 g of octadecene (1-octadecene) were mixed. The mixture was stirred for 30 minutes under reduced pressure at room temperature to remove gas and water in the mixture. The mixture was then slowly heated to 100° C. for 15 minutes under reduced pressure, and stirred at 100° C. for 40 minutes to obtain a reaction solution. The reaction solution was slowly cooled to 50° C. under an inert atmosphere, and then 148 mg of ammonium fluoride and 10 ml of methanol in which 100 mg of sodium hydroxide was dissolved were injected into the reaction solution. Thereafter, the reaction solution was stirred at 50° C. for 40 minutes under an inert atmosphere. The reaction solution was then slowly heated to 100° C. under reduced pressure, and stirred at 100° C. for 30 minutes. Thereafter, the reaction solution was slowly heated to 300° C. for 1 hour under an inert atmosphere, and stirred at 300° C. for 1 hour and 30 minutes. Thereafter, a heater was removed, and the reaction solution was slowly cooled to room temperature, and then 60 ml of ethanol was added thereto to precipitate the formed up-conversion nanoparticle. Then, supernatant was removed, and the up-conversion nanoparticle was redispersed in 1 ml of hexane. 40 ml of ethanol was again added to the up-conversion nanoparticle solution to precipitate the particle, and supernatant was removed to finally obtain an up-conversion nanoparticle (NaYF₄:Yb³⁺,Er³⁺) (Example 3).

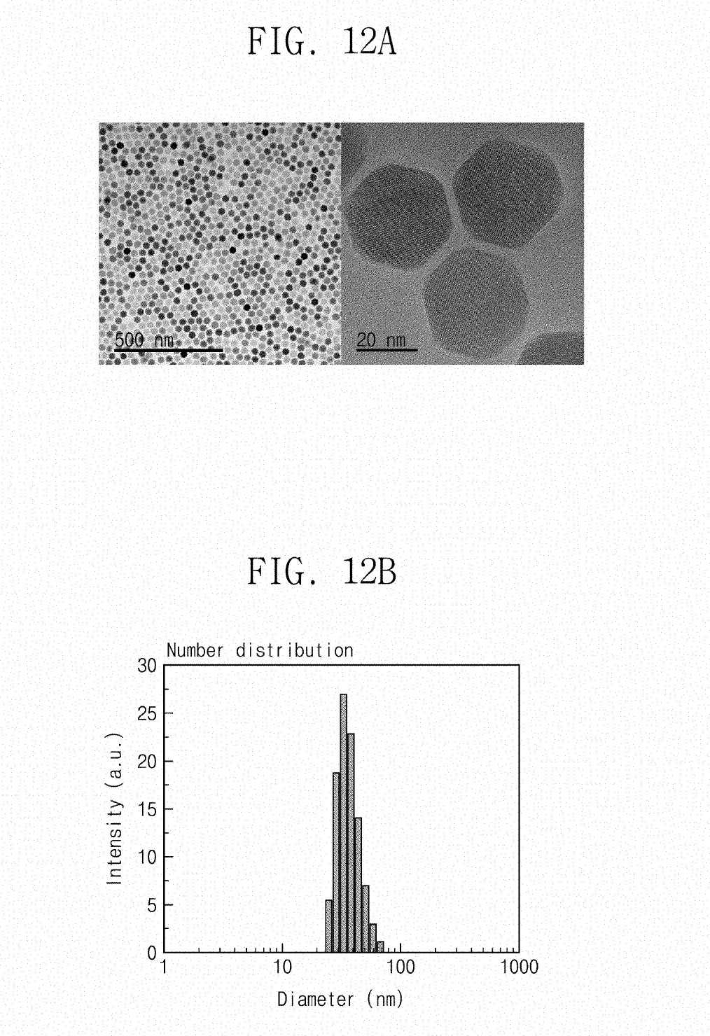

FIGS. 12A and 12B show a TEM image and a size analysis result of up-conversion nanoparticles dispersed in an organic solvent.

Referring to FIG. 12A, a TEM image of the nanoparticle of Example 3 can be seen. Through this, it was confirmed that the nanoparticle of Example 3 has a uniform size and shape. Since the nanoparticle of Example 3 is coated with oleic acid, the nanoparticle may be very well dispersed in the organic solvent. Furthermore, referring to FIG. 12B, it was confirmed that the size of the nanoparticle of Example 3 has the size distribution of 36.85±9.22 nm according to the result of DLS analysis. The up-conversion nanoparticle (NaYF₄:Yb³⁺,Er³⁺) of Example 3 is an optical contrasting agent capable of emitting light in the visible ray area by absorbing a near infrared ray of 980 nm.

### Experimental Example 5: Preparation of an Up-Conversion Nanoparticle Coated with Indocyanine Green

1 mg of the up-conversion nanoparticle (Example 3) was dispersed in 1 ml of chloroform.

After 2 mg of indocyanine green was dissolved in 4 ml of distilled water, 0.1 ml of the nanoparticle of Example 3 dispersed in chloroform was added thereto, and tip ultrasonication was performed thereon for 1 minute to prepare a first emulsion solution. The emulsion solution was vigorously stirred for 5 minutes until all the chloroform therein was volatilized and removed. The stirred solution was centrifuged to precipitate the nanoparticle of Example 3, supernatant was removed, and then distilled water was added thereto again. The process was repeated 4-5 times until the indocyanine green in the solution was removed so that the solution became transparent. The finally obtained up-conversion nanoparticle coated with indocyanine green was dispersed in 1 ml of distilled water (Example 4).

It was confirmed that the nanoparticle of Example 4, unlike the up-conversion nanoparticle of Example 3, was well dispersed in the aqueous phase without an aggregation phenomenon. The phase of the up-conversion nanoparticle was also converted to hydrophilic through the indocyanine green which is an amphiphilic organic dye.

### Example 6: Analysis of the Characteristics of an Up-Conversion Nanoparticle Coated with Indocyanine Green

The nanoparticle of Example 4 was spin coated on a slide glass to prepare a sample. 980 nm laser was irradiated on the nanoparticle of Example 4, and the up-conversion PL (photoluminescence) signal in the visible ray area was measured using a 700 nm short pass filter. 785 nm laser was irradiated on the nanoparticle of the Example 4 in in the same position, and the fluorescence signal of the indocyanine green was measured using an 830 nm long pass filter.

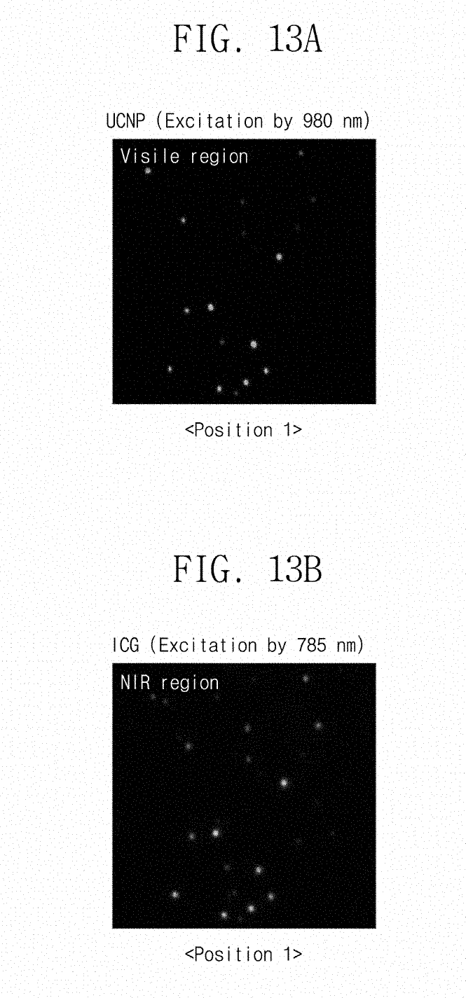

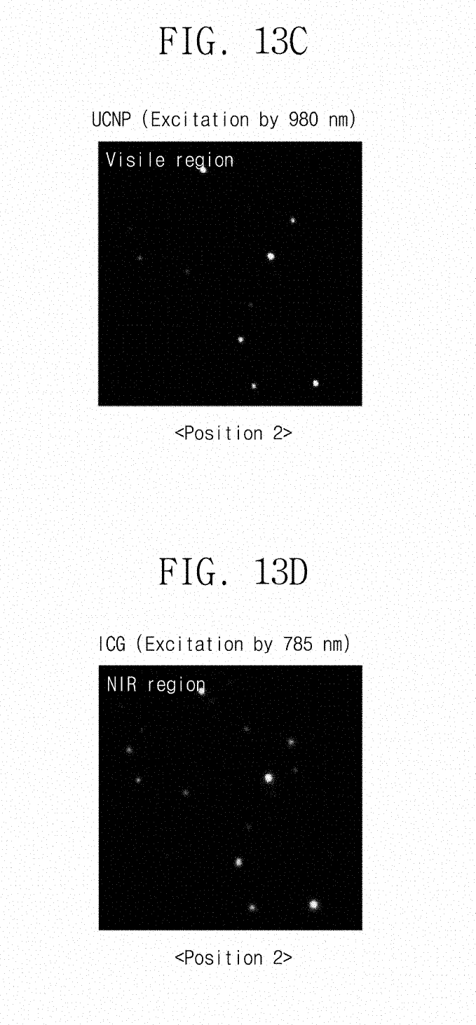

FIGS. 13A and 13C are photoluminescence (PL) images (UCNP) of an up-conversion nanoparticle coated with indocyanine green. FIGS. 13B and 13D are fluorescence images (ICG) of an up-conversion nanoparticle coated with indocyanine green. FIGS. 13A and 13B represent the same first position (Position 1), and FIGS. 13C and 13D represent the same second position (Position 2).

Referring to FIGS. 13A to 13D, both the PL and fluorescence signals of the nanoparticle of Example 4 are well displayed. Through this, it was confirmed that indocyanine green coating was well formed on the surface of the nanoparticle of Example 4. Also, in the case of the nanoparticle of Example 4, a dual color optical contrasting may be possible through a combination of two optical contrasting agents (up-conversion, and fluorescence). That is, selective observation at two specific wavelengths may be possible using a single particle.

A slide glass on which the nanoparticle of Example 4 was spin coated thereon was prepared. A region of the slide glass was selected, and was continuously irradiated by 785 nm laser for 30 minutes.

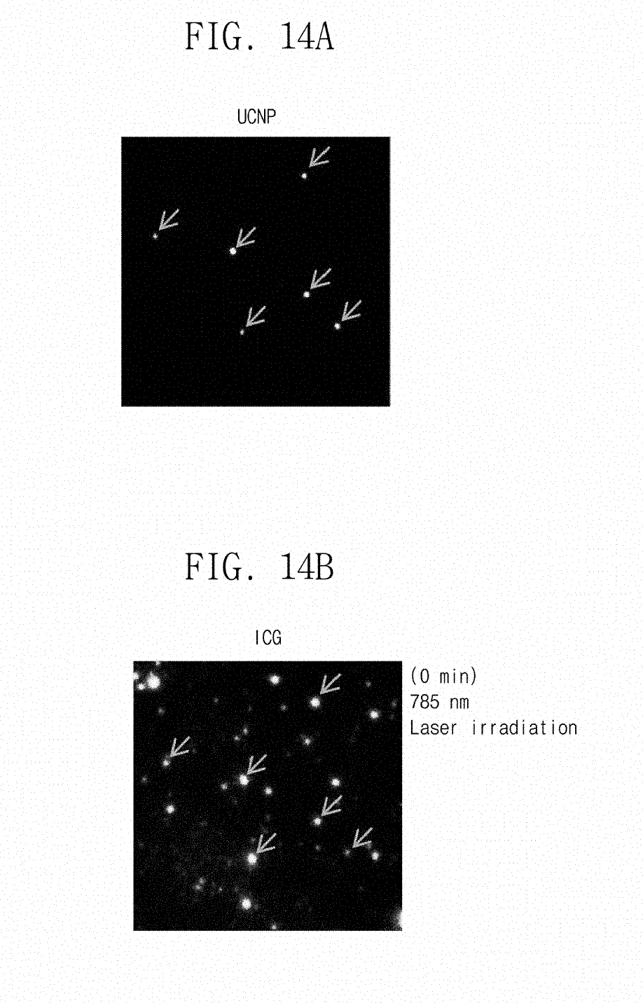

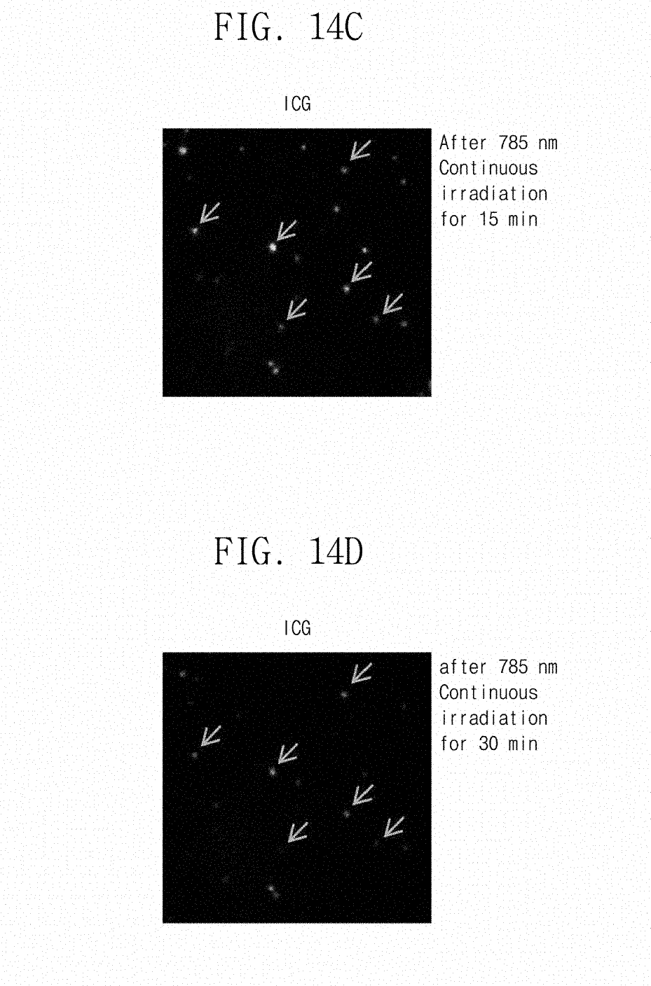

FIGS. 14A to 14D are a single particle fluorescence images showing the optical stability of an up-conversion nanoparticle coated with indocyanine green. FIG. 14A shows an up-conversion signal, and FIGS. 14B to 14D show fluorescence signals of indocyanine green over time

Referring to FIGS. 14A to 14D, when the up-conversion PL signal (FIG. 14A) and the fluorescence signals (FIGS. 14B to 14D) over time were compared, it was confirmed that the fluorescence signal of indocyanine green continued to appear even after 30 minutes of laser irradiation in the positions on which up-conversion nanoparticles are present (yellow arrows). On the other hand, in the position on which nanoparticles are not present, it was confirmed that the fluorescence signal of indocyanine green were relatively much reduced or disappeared after 30 minutes. This may indicate that the optical stability is increased by coating the indocyanine green on the nanoparticle. As described above, the hydrophobic portion of indocyanine green interacts with the surface of the nanoparticle and is combined therewith, thereby being in a structurally bound (non-flexible) state. Such a state may reduce the photobleaching phenomenon due to a structural change of the indocyanine green, so that optical stability may be increased.

The inventive concept may provide a new aspect of phase conversion method in that an amphiphilic organic dye is used as interface material which is a medium for phase conversion. Furthermore, the utilization as a fluorescence contrasting agent may be great in that the fluorescence stability of the amphiphilic organic dyes adsorbed on the surface of the particle may be increased.

1. A hydrophilic particle comprising:
a hydrophobic particle; and
and amphiphilic organic dye directly absorbed on a surface of the hydrophobic particle,
wherein the hydrophobic particle comprises a center particle, and a hydrophobic ligand covering a surface of the center particle,
wherein the amphiphilic organic dye is combined with the hydrophobic ligand by a hydrophobic interaction, and
wherein the hydrophilic particle has a surface zeta potential lower than a surface zeta potential of the amphiphilic organic dye.

2. The hydrophilic particle of claim 1, wherein the center particle comprises a transition metal oxide, and
wherein the hydrophobic ligand comprises a fatty acid.

3. The hydrophobic particle of claim 2, wherein the transition metal oxide is selected from the group consisting of iron oxide, manganese oxide, titanium oxide, nickel oxide, cobalt oxide, zinc oxide, ceria, and gadolinium oxide.

4. The hydrophobic particle of claim 2, wherein the fatty acid is selected from the group consisting of oleic acid, laurate acid, palmitic acid, linoleic acid, and stearic acid.

5. The hydrophilic particle of claim 1, wherein the center particle is an up-conversion particle, and
wherein the hydrophobic ligand comprises a fatty acid.

6. The hydrophilic particle of claim 5, wherein the up-conversion particle is selected from the group consisting of NaYF₄:Yb³⁺,Er³⁺, NaYF₄:Yb³⁺,Tm³⁺, NaGdF₄: Yb³⁺,Er⁴⁺, NaGdF₄:Yb³⁺,Tm³⁺, NaYF₄:Yb³⁺,Er³⁺/NaGdF₄, NaYF₄:Yb³⁺,Tm³⁺/NaGdF₄, NaGdF₄:Yb³⁺,Tm³⁺/NaGdF₄, and NaGdF₄:Yb³⁺,Er³⁺/NaGdF₄.

7. The hydrophilic particle of claim 5, wherein the fatty acid is selected from the group consisting of oleic acid, laurate acid, palmitic acid, linoleic acid, and stearic acid.

8. The hydrophilic particle of claim 1, wherein the amphiphilic organic dye is selected from the group consisting of rhodamine, BODIPY, Alexa Fluor, fluorescein, cyanine, phtahlocyanine, an azo-group dye, a ruthenium-based dye, and derivatives thereof.

9. The hydrophilic particle of claim 1, wherein the amphiphilic organic dye comprises, in the molecule thereof, a hydrophilic group selected from the group consisting of a carboxyl group, a sulfonic acid group, a phosphonic acid group, an amine group, and an alcohol group, and a hydrophobic group selected from the group consisting of an aromatic hydrocarbon and an aliphatic hydrocarbon.

10. The hydrophilic particle of claim 1, wherein the surface zeta potential of the amphiphilic organic dye is a value measured when the amphiphilic organic dye is present alone.

11. The hydrophilic particle of claim 1, wherein the surface zeta potential of the hydrophilic particle is a negative charge.

12. The hydrophilic particle of claim 1, wherein an average diameter of the hydrophilic particle is greater than an average diameter of the hydrophobic particle.

13. A method for manufacturing a hydrophilic particle comprising:
preparing a hydrophobic particle dispersed in an organic phase; and
mixing the hydrophobic particle in the organic phase with an amphiphilic organic dye in an aqueous phase to form a hydrophilic particle,
wherein the amphiphilic organic dye is directly absorbed on a surface of the hydrophobic particle to phase-convert the hydrophobic particle to the hydrophilic particle dispersed in the aqueous phase.

14. The method of claim 13, wherein the mixing of the hydrophobic particle and the amphiphilic organic dye comprises:
adding the hydrophobic particle in the organic phase to the amphiphilic organic dye in the aqueous phase; and
ultrasonicating a mixture of the hydrophobic particle and the amphiphilic organic dye to form a water-in-oil (O/W) emulsion.

15. The method of claim 13, wherein the organic phase comprises an organic solvent selected from the group consisting of chloroform, cyclohexane, hexane, heptane, octane, isooctane, nonane, decane, and toluene.

16. The method of claim 13, wherein the hydrophobic particle comprises a hydrophobic ligand on a surface thereof, and
wherein the amphiphilic organic dye is combined to the hydrophobic ligand by a hydrophobic interaction.

17. The method of claim 13 further comprising, after forming the hydrophilic particle, evaporating the organic solvent forming the organic phase.

18. A contrasting agent comprising a hydrophilic particle, wherein the hydrophilic particle comprises:
a hydrophobic particle; and
an amphiphilic organic dye directly adsorbed on a surface of the hydrophobic particle, and
wherein a surface zeta potential of the hydrophilic particle is lower than a surface zeta potential of the amphiphilic organic dye.

19. The contrasting agent of claim 18 is used for magnetic resonance imaging, optical imaging, or magnetic resonance imaging and optical imaging.
